(19) United States
(12) Patent Application Publication
(10) Pub. No.: US 2015/0025310 A1
(43) Pub. Date: Jan. 22, 2015

(54) MEDICAL APPLICATOR

(71) Applicant: OI MEDICAL LIMITED, Sheffield (GB)

(72) Inventors: John Sanders Everingham, Fulwood (GB); Gilbert Marcus Filshie, Mapperley Park (GB)

(21) Appl. No.: 14/383,299

(22) PCT Filed: Mar. 6, 2013
(86) PCT No.: PCT/GB2013/000094

§ 371 Date: Sep. 5, 2014

(30) Foreign Application Priority Data
Mar. 7, 2012 (GB) 1203994.7

(51) Int. Cl. A61F 6/22; A61B 1/00; A61B 1/04; A61B 1/005; A61B 1/015; A61B 17/12; A61B 1/07

(52) U.S. Cl. CPC A61F 6/225; A61B 17/12031; A61B 17/12159; A61B 17/12013; A61B 1/00087; A61B 1/00133; A61B 1/07; A61B 1/00101; A61B 1/0057; A61B 1/00006; A61B 1/015; A61B 1/04; A61B 1/00045; A61B 2017/12095; A61B 2017/00398
USPC 600/103; 600/106

(57) ABSTRACT

An applicator for use in the fitting of an occlusive plug of the screw in kind to a fallopian tube, said applicator comprising: a body member; an eyepiece fitted to one end of the body member; an elongate probe carried by, and projecting from the other end of the body member; a fibre optic cable extending from the eye-piece along the interior of the probe to a terminal end of the probe distal from the body member; a nose piece movably mounted at said distal end of the probe and adapted to releasably receive a rear, drive end of an occlusive plug; a mechanical connection extending between the body member and the nose-piece whereby the practitioner may, within limits, vary the angle of the nose-piece with respect to its carrier tube; and a rotational drive means extending between the body member and the nose-piece to apply torque & hence rotational movement to an occlusive plug temporarily held by the nose-piece.

### FIELD OF THE INVENTION

This invention relates to a medical applicator for applying an occlusive plug to a fallopian tube of a female.

### BACKGROUND OF THE INVENTION

For female sterilization the known Filshie procedure using the known Filshie clip system is recognised as the leading surgical procedure worldwide, accounting for over 80% of female sterilisations in England and Canada, and having a low failure rate of around 0.27%. The Filshie clip system involves making an incision in the patient's abdomen and fitting a removable clip around each of the patient's fallopian tubes and therefore requires post operative care and a hospital stay.

As an alternative to the Filshie clip, the plugging of fallopian tubes as a medical procedure has been known both in principle and in practice for some years as a means of sterilisation. These procedures avoid abdominal surgery and involve the use of a medical applicator inserted in to the patient's womb via the vagina, and are therefore less traumatic than abdominal surgery. Examples of the techniques are known in WO 97/12569, and WO2007/072004.

WO97/12569 discloses a hysteroscope used for insertion of a self tapping screw occlusive plug into the fallopian tubes, and using a flexible tube applicator having an octagonal head connector for attaching to an occlusive plug.

However, the fitting of a plug into a fallopian tube is not without its difficulties despite the availability of hysteroscopic vision to aid the medical practitioner.

### OBJECT OF THE INVENTION

An object of the present invention is the provision of an improved applicator for use in the fitting of an occlusive plug of the screw-in kind to a fallopian tube.

### SUMMARY OF THE INVENTION

According to one aspect of the present invention, there is provided a medical applicator for use in female sterilisation of a fallopian tube, said applicator comprising:

a body member;

an elongate probe carried by, and projecting from one end of the body member;

a viewing means extending along the probe to an end of the probe distal from the body member;

a connector piece movably mounted at said distal end of the probe;

means for varying the position of the connector with respect to a main length of said probe; and

a rotational drive means extending between the body member and the connector to apply rotational movement to said connector.

According to a second aspect there is provided an applicator for use in the fitting of an occlusive plug of the screw in type to a fallopian tube, said applicator comprising:

an elongate probe member, said probe member comprising:

an elongate hollow tubular member;

an elongate flexible drive shaft extending along said tubular member to a distal end of said probe;

wherein said hollow tubular member, comprises at a distal end, a deflectable nose portion which can be controllably directed in a direction making an angle in the range 0 to 60 degrees to a main length direction of said probe.

According to a third aspect there is provided an applicator for use in the fitting of an occlusive plug of the screw in type to a fallopian tube, said applicator comprising:

an elongate probe member;

a drive shaft extending along said probe member;

an electric stepper motor connected to said drive shaft; and

a controller for controlling operation of said stepper motor;

wherein said electric stepper motor is configured to drive said drive shaft in a clockwise or an anti-clockwise direction.

According to a fourth aspect there is provided an applicator for use in the fitting of an occlusive plug said applicator comprising:

an elongate probe;

a connector at a distal end of said probe, for connecting to an occlusive plug;

a fluid tube extending along said probe for carrying fluid to a distal end of said probe;

a fibre optic viewing cable extending along said probe for viewing at a distal end of said probe from a viewing position at a near end of said probe;

an fibre optic light transmission cable for conducting light from said near end of the probe to said distal end of the probe for illuminating said distal end of said probe;

wherein said fluid channel, said fibre optic viewing cable and said fibre optic light transmitting cable are all formed into a single unitary flexible cable extending along a length of said probe.

According to a fifth aspect there is provided a method of using an applicator, said applicator comprising:

a body member;

an elongate probe carried by, and projecting from one end of the body member;

a viewing means extending along the probe to an end of the probe distal from the body member;

a connector piece movably mounted at said distal end of the probe;

means for varying the position of the connector with respect to a main length of said probe; and

a rotational drive means extending between the body member and the connector to apply rotational movement to said connector,

said method comprising:

fitting an occlusive plug to said connector;

inserting said probe into a neck of a womb;

deflecting said distal end of said probe to point towards a cornu region;

using said viewing means to view a region around said distal end of said probe;

locating a forward end of said occlusive plug at an ostium;

operating said rotational drive means to rotate said connector a predetermined number of revolutions to drive said occlusive plug into said ostium; and

detaching said connector from said occlusive plug.

According to a sixth aspect there is provided a method of female sterilisation using an elongate uterine probe, said uterine probe comprising:

(i) a viewing means extending along the probe to a distal end of the probe;

(ii) a connector piece movably mounted at said distal end of the probe for connecting to an occlusive plug;

(iii) means for varying the position of the connector with respect to a main length of said probe; and

(iv) a rotational drive means operable to apply rotational movement to said connector;

said method comprising:

fitting an occlusive plug to said connector at said distal end of said probe;

inserting said probe into a neck of a womb;

deflecting said distal end of said probe within a uterine cavity, to point towards a cornu region;

using said viewing means to view a region around said distal end of said probe;

locating a forward end of said occlusive plug at an ostium;

operating said rotational drive means to rotate said connector to drive said occlusive plug into said ostium; and

detaching said connector from said occlusive plug.

According to a seventh aspect there is provided a method of reversing a female sterilisation caused by an occlusive plug positioned in a fallopian tube of a female, using a uterine probe, said probe comprising:

(i) a viewing means extending along the probe to a distal end of the probe;

(ii) a connector piece movably mounted at said distal end of the probe for connecting to an occlusive plug;

(iii) means for varying the position of the said distal end of the probe with respect to a main length of said probe; and

(iv) a rotational drive means operable to apply rotational movement to said connector;

said method comprising:

inserting said probe into a neck of a womb;

deflecting said distal end of said probe within a uterine cavity, to point said distal end towards a cornu region;

using said viewing means to view a region around said distal end of said probe to locate an end of an occlusive plug protruding from an ostium;

locating said connector at said protruding end of said occlusive plug and attaching said connector to said protruding end;

operating said rotational drive means to rotate said connector to withdraw said occlusive plug from said ostium.

The invention includes an applicator for use in the fitting of an occlusive plug of the screw-in kind to a fallopian tube comprising:
(i) an elongate body member;
(ii) an eyepiece fitted to one end of the body member;
(iii) a hollow probe carried by, and projecting from, the other end of the body member;
(iv) a fibre optic cable extending from the eye-piece along the interior of the probe to the terminal end of the probe distal from the body member;
(v) a nose piece movably mounted with respect to one end of a carrier tube which is located internally of the probe, and adapted to releasably receive a rear, drive end of an occlusive plug;
(vi) a mechanical connection extending between the body member and the nose-piece whereby the practitioner may, within limits, vary the angle of the nose-piece with respect to its carrier tube; and
(vii) a rotational drive means extending between the body member and the nose-piece to apply torque & hence rotational movement to an occlusive plug temporarily held by the nose-piece.

Other aspects are as set out in the claims herein.

### Advantages of the Invention

Specific embodiment applicators disclosed herein may provide a single instrument with which a practitioner may, aided by hysteroscopic vision, accurately locate a leading end of a screw type occlusive plug in the lumen of a fallopian tube, by firstly applying any angular adjustment of the plug that may be necessary to locate the nose of a plug at an ostium, and when so located, by rotating the plug to screw it further into the fallopian tube, for example by a distance of up to 20 mm, so that the plug effects a self-retaining engagement of the lumen.

### BRIEF DESCRIPTION OF THE DRAWINGS

For a better understanding of the invention and to show how the same may be carried into effect, there will now be described by way of example only, specific embodiments, methods and processes according to the present invention with reference to the accompanying drawings in which:

FIG. 1 herein illustrates schematically one example of an applicator in accordance with a specific embodiment of the present invention;

FIG. 2 herein illustrates schematically in cut away view the applicator of FIG. 1 herein;

FIG. 3 herein, shows the applicator in view from above;

FIG. 4 herein shows the applicator in view from one side;

FIG. 5 herein shows the applicator in view from the front end;

FIG. 6 herein shows a close up view of the nose of the applicator, showing a connector for attaching an occlusive plug;

FIG. 7 herein shows the probe of the applicator in a first configuration carrying an occlusive plug, for initial insertion into a patient, having a cover sheath extended over the nose of the probe;

FIG. 8 herein shows the probe in a configuration for a second phase of operation, in which the sheath is retracted to expose the nose portion;

FIG. 9 herein shows the probe in a third configuration, in a third phase of operation in which the nose of the probe is deflected from a straight ahead position;

FIG. 10 shows the probe in a fourth configuration in which the probe is disengaged from an occlusive plug; and

FIG. 11 herein shows schematically a view as seen through the eye piece of the applicator, of an occlusive plug attached to the end of the applicator whilst the applicator is positioned within the womb of a female patient.

### DETAILED DESCRIPTION OF THE EMBODIMENTS

There will now be described by way of example a specific mode contemplated by the inventors. In the following description numerous specific details are set forth in order to provide a thorough understanding. It will be apparent however, to one skilled in the art, that the present invention may be practiced without limitation to these specific details. In other instances, well known methods and structures have not been described in detail so as not to unnecessarily obscure the description.

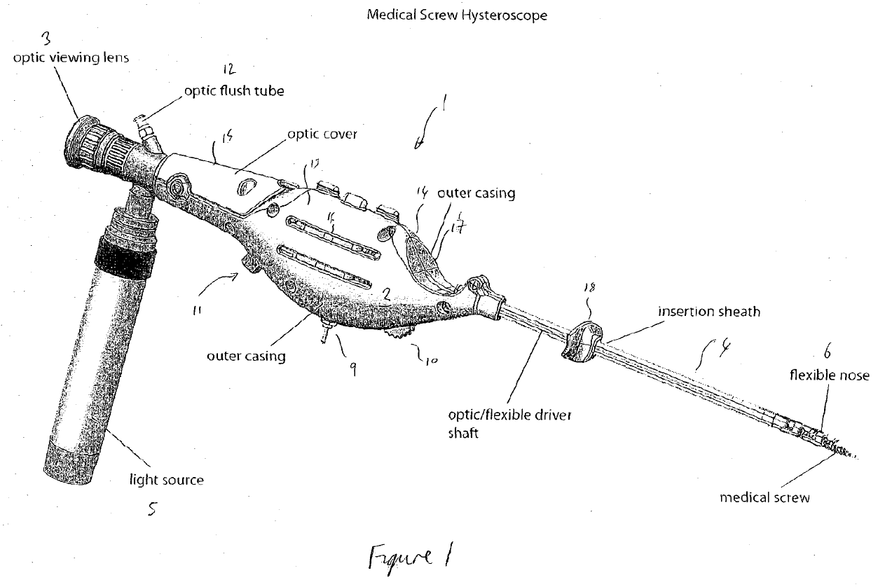

Referring to FIG. 1 herein, there is shown in perspective external view an applicator for fitting an occlusive plug of the screw in type into a fallopian tube for the purpose of reversible female sterilization.

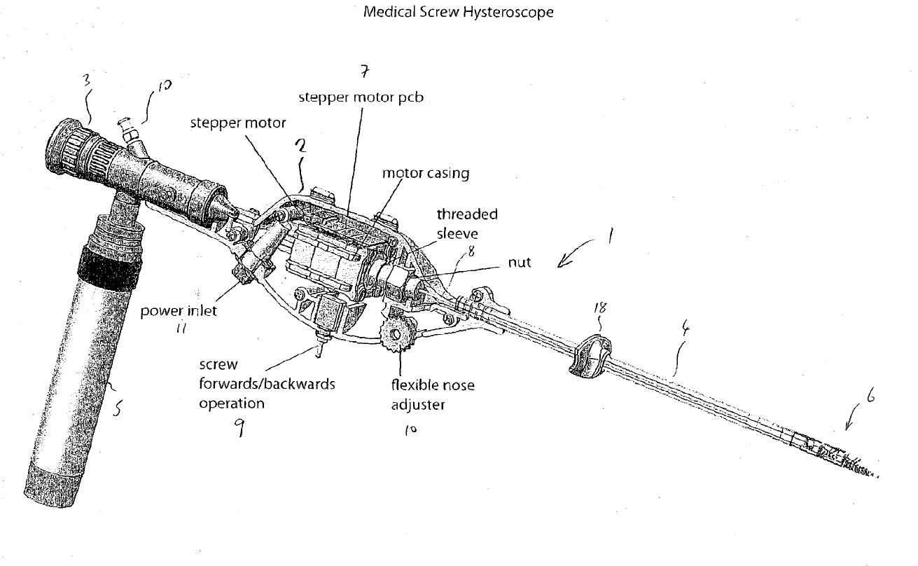

Referring to FIG. 2 herein, there is shown in cut away view an applicator for fitting an occlusive plug of the screw in kind, to a fallopian tube.

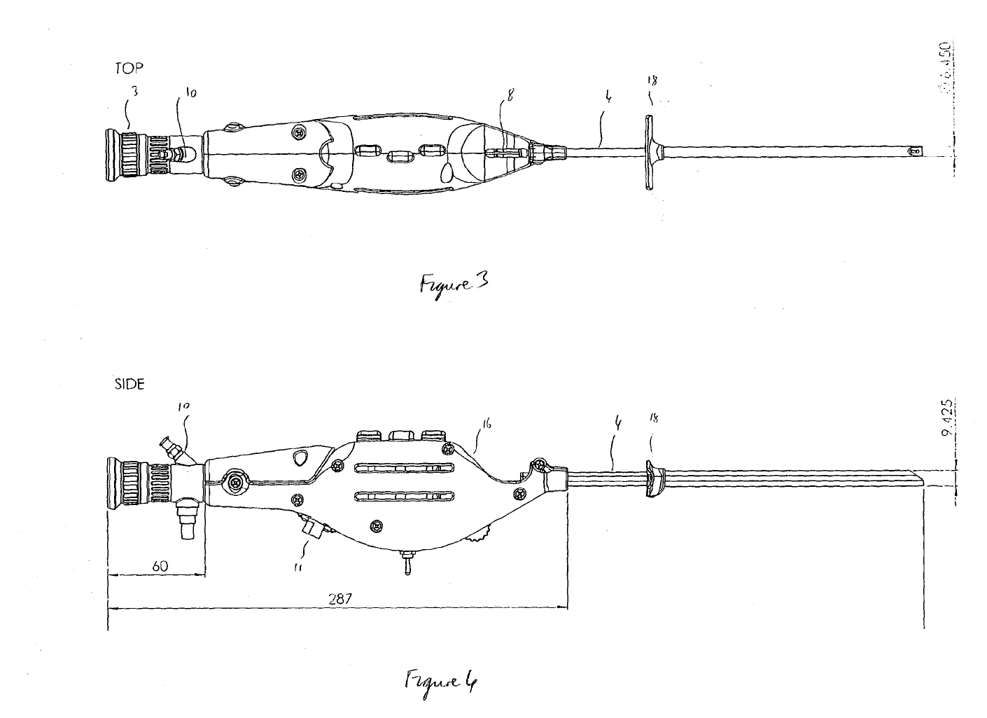

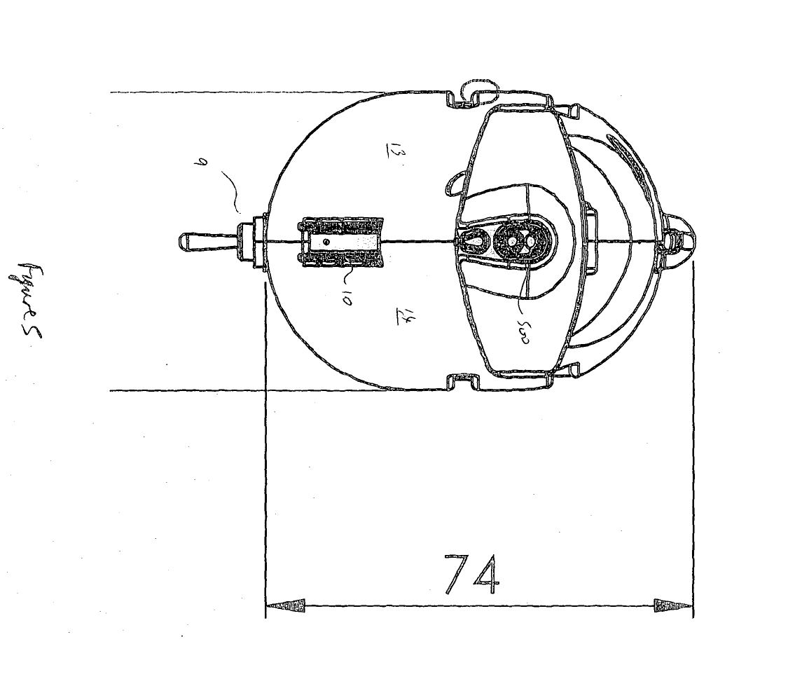

Referring to FIGS. 3 and 4 herein, there is shown the applicator in views from above and one side respectively.

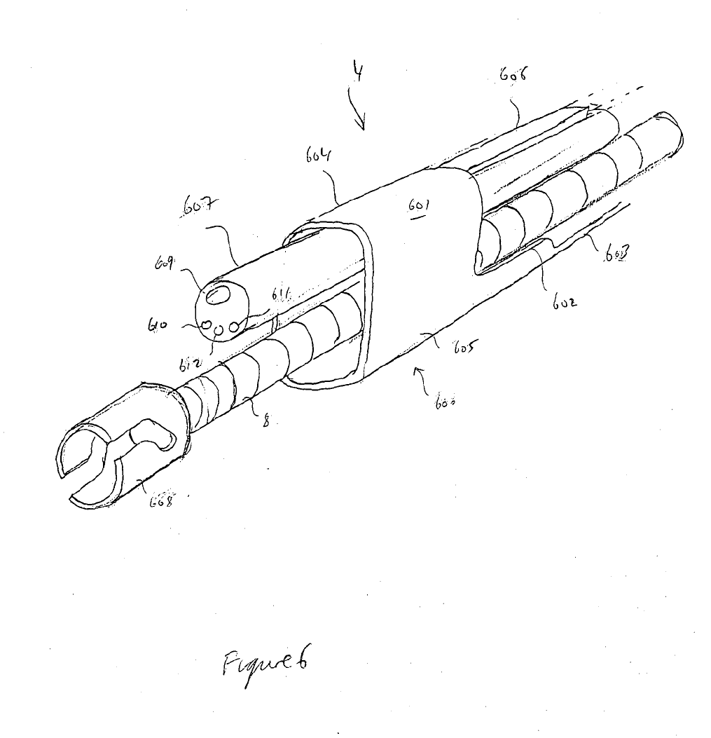

Referring to FIG. 5 herein there is shown the applicator in view from the front.

As shown on FIGS. 1 and 2, the applicator 1 comprises an elongate body member 2 having a molded plastics outer casing; an eye-piece 3 at a rear end of the body; an elongate hollow probe 4 at a front end of the body; an optional battery pack and fight source 5 positioned towards the rear of the body; a universal light connector for accepting either the battery pack & light source, or a surgical theatre light source; a bendable nose assembly 6 positioned at a forward end of the probe 4, for inserting a plug into a fallopian tube; a fibre optic cable extending from the eye piece, along the probe member to the nose assembly, for viewing inside a womb; a light conducting fibre optic cable for transmitting light from the light source to the end of the probe for illuminating an area around the end of the probe; a fluid tube for carrying fluid along the probe and emitting fluid in the region around the end of the probe; in a best mode embodiment, the viewing fibre optic, illuminating fibre optic and fluid conducting tube being formed into a single flexible cable extending along the length of the probe to the end of the probe; an electric stepper motor 7 for rotating a plug relative to the probe; optionally, a gearbox driven by the stepper motor; a flexible drive shaft 8 extending along the probe and between the gearbox and a distal end of the probe, and driven in a reverse or forwards direction by the stepper motor, optionally via the gearbox or in a preferred embodiment directly; a forwards/reverse switch 9 for operation of the stepper motor to turn the drive shaft in a forwards or reverse direction; an angular adjustment mechanism 10 for manually adjusting an angle of the nose relative to the probe 4; a power inlet 11 for connection to an external electrical power supply for powering the stepper motor; and a fluid inlet 12 for introducing fluid to flush around the nose of the device inside a patient's womb so as to distend the uterine cavity.

Casing

The casing 1 is formed of two approximately mirror image shell halves 13, 14 which surround the front end of the eyepiece, and other internal components such as the switch 9, stepper motor 7, stepper motor printed circuit board, and part of the fluid tubes and fibre optic cables. At the rear of the casing is provided a cover portion 15 for covering the optics at the front of the eyepiece, which is removable separate from the casing shell halves 13, 14 for inspection of the front end of the eye piece, and the end of the viewing fibre optic. The casing shell halves 13, 14 may be provided with slots or apertures 16, to allow air to flow through the casing for cooling of the step motor 7.

A front portion 17 of the outer casing 1 may provide a curved trough shaped recess which allows viewing of the near or proximal end of the probe, and in particular to allow viewing of the flexible drive shaft, and an elongate spring which extends along the length of the probe for bending the nose end of the probe 6. The flexible drive shaft 8 protrudes through an aperture in the trough portion of the casing, before entering an open end of the tubular probe 4. This allows the operator to visually check that the drive shaft is rotating, without the need to remove the probe from the patient's body.

In other embodiments, the trough 16 at the front end of the casing may be covered with a transparent see through plastics cover.

In a preferred embodiment, the casing has a length, excluding eye piece and excluding the probe member in the range 200 mm to 280 mm, and preferably of the order of 267 mm; a maximum width in the range 40 mm to 60 mm, and preferably of the order of 50 mm; and a maximum height in the range 58 mm to 78 mm, and preferably of the order of 68 mm.

Preferably the two shell halves of the casing are connected together by a plurality of screws. Similarly, the rear cover 15 is retained to the casing shell halves by a pair of small screws.

Probe

The probe 4 comprises an elongate rigid tubular member 8, having a rotatable drive rod passing there through, which can rotate relative to the rigid tube 8. A distal end of the drive member is fitted with a bayonet fitting type socket connector 9, comprising a substantially cylindrical hollow tubular member having a pair of diametrically opposed slots, for engaging with a substantially cylindrical end of the plug, where the end of the plug has a pair of radially extending bayonet type protrusions, which engage with the slots in the hollow cylindrical end of the bayonet connector of the probe. In use, the plug can be inserted into a fallopian tube on the end of the probe, rotated in a first direction to drive the plug along the fallopian tube, for example by distance of up to 20 mm, and then the rotational direction of the drive means is reversed, to disengage the bayonet socket from the end of the plug, thereby leaving the plug in situ within the fallopian tube. Subsequently, the probe member can be removed from the patient's body.

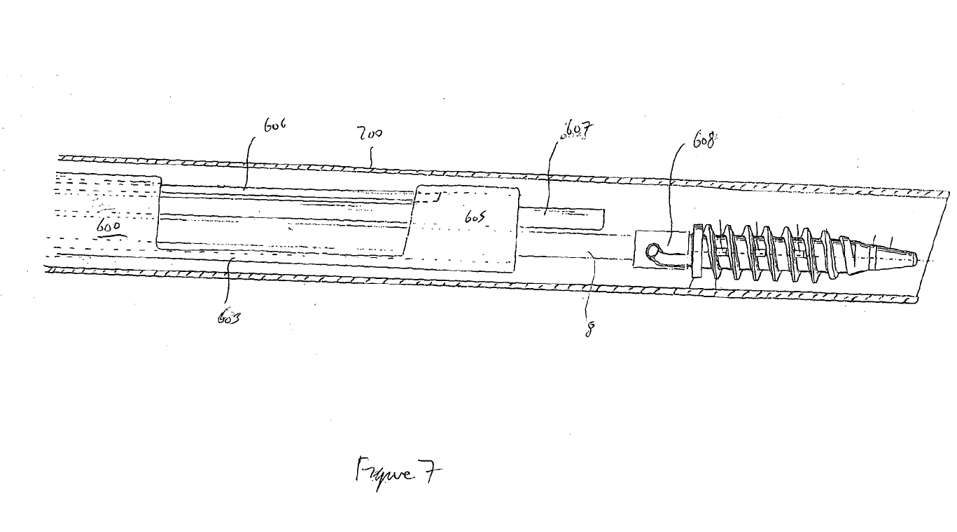

Referring to FIG. 6 herein, there is illustrated schematically in perspective view a distal end of the probe 4.

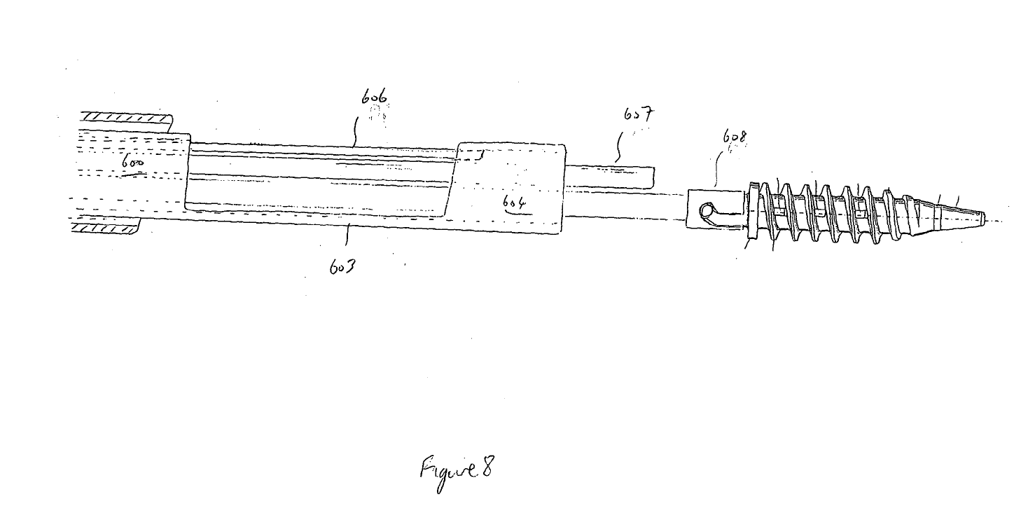

Referring to FIG. 7 herein, there is shown in side view the distal end of the probe, covered by an outer sheath. The outer sheath is slidable over the nose of the probe so as to protect the probe and attached plug when entering or being withdrawn from the uterine cavity. The end of the sheath has a chamfered end to assist in insertion into and removal from the uterine cavity.

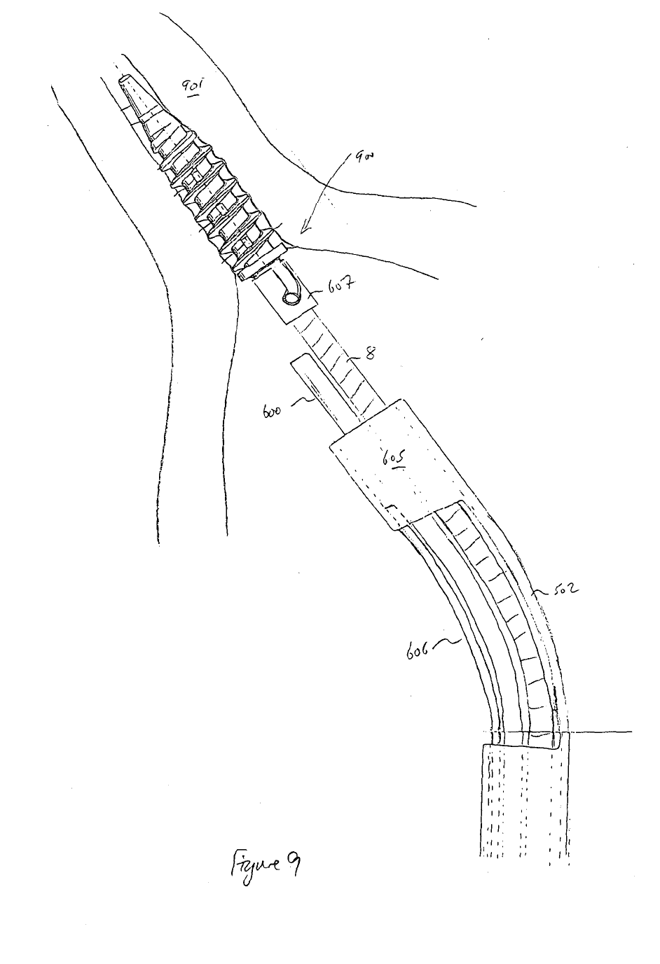

Referring to FIG. 8 herein, there is shown the distal end of the probe, with the outer sheath retracted.

The tubular wall 600 of the probe is substantially rectangular in cross section, but having rounded corners 601. At the distal end of the tube comprising the nose portion of the probe, the tube has a window portion 602 whereby 3 sides of the tube are removed over a distance of around 15 mm to 35 mm, and leaving only one side 603 of the tube present. At the distal end of the tube, the tube provides a loop 604, through which the drive shaft 8 and multi-function cable 6 pass.

Secured to the end loop 604 of the tube 600, a strip 605 is provided, being preferably a leaf spring which is flexible enough to bend to allow the nose to bend to an angle of up to 60°, but rigid enough to be able to push or pull the loop 604 on the end of the probe so that the end loop 604 can be deflected, either side of a straight ahead or axial direction of the main probe tubular body. The leaf spring is located substantially parallel with and spaced apart from the exposed tube side 603, so that the leaf spring faces opposite the sidewall 603 across a gap.

Both the multi-function cable 607, and the flexible drive rod 8 pass through the hollow tube 600 and protrude from the distal end of the tube. As shown in FIG. 6, the flexible drive rod 8 preferably comprises a normally cylindrical elongate helically wound tube, which allows the distal end of the drive shaft to be self supporting, but also allows enough flexibility for the drive shaft to bend whilst under rotation either clockwise or anti-clockwise, whilst being retained within the tube and within the tubular loop 604 at the end of the tube,

Referring to FIG. 7 herein, there is illustrated the end of the probe, having an occlusive plug fitted to the bayonet fixing 608 at the end of the drive shaft 8, and with the outer sheath or sleeve 700 slid forward to protect the nose. In this configuration, the sheath protects the occlusive plug and the drive shaft, multi-function cable 607 and loop 604 from catching or snagging on the sides of the vagina or womb during insertion and thereby prevents the occlusive plug from detaching from the end connector 608. In a preferred embodiment, the end connector 608 comprises a hollow cylindrical tube having a pair of bayonet fixing slots, to cooperate and engage with a pair of protruding lugs on the end of a cylindrical end of an occlusive plug. However, in other embodiments, the connector 608 may comprise a cylindrical socket having an elongate hexagonal or square recess to cooperate with a hexagonal or square end of an occlusive plug,

Referring to FIG. 8 herein, there is shown the end of the probe 6 in a second configuration, whereby the outer sheath 700 is retracted, thereby exposing the window portion of the tube 600 and exposing the occlusive plug and connector 608.

The probe comprises a hollow tube 600, manufactured from a bendable material, for example medical grade polycarbonate, or a metal alloy, the material being selected such that when the tube is fully integral having a solid tubular surface, the tube is substantially rigid, but will allow a slight degree of flexing by a few degrees over its length, and such that at a distal end of the tube, with a section of tube wall removed such that only one side wall portion 603 remains, the material will flex enough to bend over an angle of up to 60° with respect to the inline position, without the material of the tube fracturing.

Multi-Function Cable

In a best mode, running in parallel inside the rigid tubular member, there is provided a multi function cable 609 which consists of a tubular passage for transporting fluid from the fluid flush inlet 13 to the distal end of the probe such that a clear fluid can be inserted into a patient's womb at the end of the probe, thereby giving expansion of the womb cavity and enabling the end of the probe to be more easily moved around within the womb; a light conducting fibre optic cable or cable bundle 610,611, for transmitting light from the light source to the end of the probe and illuminating the area of the womb within the immediate vicinity of the probe nose; and a fibre optic viewing cable 612 arranged to view in a direction along the end of the nose, so as to see the occlusive plug and the womb wall in the immediate area in front of the plug via the eye piece 3.

Referring to FIG. 6 herein, there is illustrated schematically the end of the probe 4, showing in detail the end of the multi-function cable 600. The multi-function cable comprises a flexible plastics, substantially cylindrical elongate body, having formed therein a first tubular substantially cylindrical passage 600, which is used for delivery of fluid to the end of the probe, one or more multi-mode fibre optic cables 610, 611 which are coupled at a near end to the light source 5, for illuminating the distal end of the drive shaft 8; and a viewing optical fibre 612, comprising a bundle of one or more multi-mode glass or plastics optic fibre cables, and optionally a small dimension lense attached to the distal end of the cable, for viewing a distal end of the drive shaft 8, and a region in front of the nose. In the best mode, the substantially cylindrical multi-function cable 607 comprises a substantially cylindrical body molded around the optical fibre cables, and having hollow central passage 609 there-through. The multi-function cable is flexible, so that the cable can bend as the nose of the probe is deflected.

Slideable Insertion Sheath

Along the probe, and surrounding the probe is provided a slidable protective sheath 700 comprising a hollow tube surrounding the outside of the probe. The function of the tubular sheath is to aid insertion of the probe into the patient's womb, whilst at the same time providing a covering for the nose portion of the probe which holds the occlusive plug. Since the occlusive plug is relatively small and held in place by a connector, the sheath protects the plug against detachment from the end of the probe during the initial insertion of the probe through the cervix and through towards the ostium of the patient. The distal end of the tubular sheath has a chamfered end to aid insertion and removal, and so as to provide a relatively blunt end to the probe during initial insertion of the applicator.

The sheath can be slid back to expose the nose of the probe when the probe is in situ in the womb, and sliding the sheath back then allows deflection of the nose portion relative to the main elongate axis direction of the probe.

The end of the sheath nearest or proximal to the body 2 of the applicator is provided with a plate 18 which allows the medical practitioner to grasp the end of the sheath and either withdraw or extend the sheath relative to the end of the probe, that is, to slide the sheath backwards or forward along the probe.

The plate 18 also allows the medical practitioner to judge the depth of the probe within the patient's body, and to judge the position of the probe relative to the length of the probe.

On withdrawal of the probe from the patient's body, once the nose end of the probe is straightened, the sheath can be slid back over the probe end, whilst the end of the probe is still in the uterine cavity, and this protects the end of the probe against snagging on removal, and if a plug is attached, prevents the plug detaching from the probe end and becoming loose in the uterine cavity.

Drive Mechanism

The drive mechanism for rotating the bayonet socket and rotating the plug comprises the electric stepper motor 7, operated by a forward-off-reverse switch 9 located on the underside of the body; the electric motor 7 being connected via a gearbox to the flexible torsional drive rod 8 which passes through the rigid tubular member 600, and at a distal end of which is fitted the bayonet socket 608. The electric motor can be driven in a forward or a reverse direction by operation of the forwards/backwards switch, and its speed may be reduced by gearing in the optional gearbox. Preferably a gear ratio of 1:6 is provided to reduce the motor speed.

The drive electronics to the stepper motor incorporates a means to induce step wise rotation of the drive shaft so as to induce vibration at the nose assembly, so as to vibrate the bayonet fixing end of the plug from the bayonet socket end of the drive mechanism, when in situ in the fallopian tube. Hence, the medical practitioner, by using the touch switch 9 can effect various modes of operation of the drive shaft as follows:
Smooth clockwise rotation of the drive shaft, with continuously variable speed over a predetermined range. This is used for insertion of the plug into a fallopian tube. The number of revolutions per activation can be predetermined, so that activation of the switch in a forward direction makes seven clockwise revolutions. The number of revolutions may be preset to the type of plug used, and the pitch of thread on the plug.
Smooth counterclockwise rotation of the drive shaft with controllable continuously variable speed. This is used for movement of the occlusive plug into the fallopian tube, and possible for rotating the end of the bayonet connector to align with the ends of the plug for engagement with the plug end.
Stepwise clockwise angular rotations of the drive shaft at a stop-start frequency in the range 5 to 20 Hz. This is useful for fine adjustment of the plug position within the fallopian tube.
Stepwise counterclockwise angular rotations of the drive shaft at a stop-start frequency in the range 5 to 20 Hz. This is useful for disengaging the bayonet end connector of the drive shaft from the end of the plug when in situ in the ostium.
Alternating clockwise/counterclockwise rotations of the drive shaft at a direction reversal frequency in the range 5 Hz to 20 Hz. This may be useful if the plug encounters difficulty in removal from the bayonet connector and operates to rotationally “shake” the plug off the end of the drive shaft.
Variations on the alternating clockwise/anticlockwise rotation, such as two anti clockwise step rotations followed by one clockwise step rotation, or in the general case a number X anticlockwise angular movements followed by a number Y clockwise angular movements. With X and Y being variable and controlled by predetermined program.

In the general case, the electric drive to the stepper motor may be programmable by the practitioner to achieve a set of drive speeds and angular rotational movement profiles to best suit the medical conditions of patients, and optimized for accuracy and controllability of the plug rotation and manipulation within the body.

Eye Piece and Optics

Eye piece 3 comprises a substantially tubular molding of synthetic plastics material, having a plurality of lenses arranged to focus on one end of the fibre optic cable. Within the optics inside the eye piece housing, is provided a light splitter, to allow light from the light source to be either passed down the fibre optic viewing cable to illuminate the end of the nose assembly, or through one or more separate light conducting fibre optic cables. The fibre optic viewing cable preferably comprises one or more multi-mode fibres, through which an image can be viewed at the end of the nose assembly, as magnified by the lenses and prisms within the optics set within the eye piece housing.

The tubular eye piece 3 is aligned with the end of the viewing fibre optic, by fitment into the rear end of the casing 1, which holds a near end of the fibre optic viewing cable. The fibre optic viewing cable itself comprises one or a plurality of multi-mode glass or plastics fibre optic cables as are known in the art. The viewing fibre optic cable has a near end which is positioned at a focal point of the eye piece 3, and a distal end positioned at the end of the probe behind the end of the flexible drive shaft, so as to allow viewing of the end of the flexible drive shaft through the eye piece. Since the optical fibre has a degree of flexibility, this allows the viewing optical fibre to bend along with the end of the nose of the probe. The distal end of the viewing optical fibre may be provided with a small dimension lens on the end of the fibre to allow light from the region around the nose portion to be focused on the end of the optical fibre for viewing.

The eye piece is optionally detachable so that a camera can be attached to the end of the optics tube instead of the eye piece, allowing the view to be seen on a monitor screen.

In some embodiments the eye piece may be removable.

Illumination System

The illumination system comprises a universal connector or socket and a focusing lens or mirror. The connector can accept either a detachable battery pack 5, which is provided at one end with a light emitting diode or incandescent light filament light source, or a known dedicated surgical theatre light source.

The light source 5 attaches to the eye piece 3 so that the light couples with the light splitter, which can be for example a prism.

In one embodiment, the light can be split by a prism and sent down the viewing fibre optic cable, so that the same fibre optic cable is used both for viewing, and for delivery of illumination to the end of the probe.

However, in a preferred embodiment and best mode of the invention, the light source is coupled to one or more independent optical fibres the specific function of which is to transmit light from the light source, along the probe member and to illuminate the end of the probe at the nose 6. In the best mode, one or more light conducting optical fibres, and a viewing optical fibre are incorporated into a single multi-function cable which extends from the eye piece to a position immediately rear of the end of the drive shaft at the end of the nose 6 of the probe 4.

Fluid Delivery

The applicator can be connected to an external fluid source via the luer type fluid connector 10 which comprises part of the housing of the optics assembly. In the best mode, where a multi-function cable is used, the cable comprises a hollow tubular passage through which a fluid, for example de-ionized water, or other clear transparent medical grade fluid can be injected. Pressurized fluid flows through the connector 10, into the tubular fluid passage of the multi-function cable and is delivered to the nose or the probe 6. When the probe is inside the body, enough fluid can be delivered to expand the womb cavity, so that the walls of the womb in the region of the ostium are expanded, thereby allowing greater freedom of movement of the end of the probe 6 within the womb cavity. Fluid may be delivered under pressure, for example using an electric pump, or under gravity pressure, for example from a drip feed bag.

Angular Adjustment of the Probe

The nose assembly can be moved angularly with respect to a main length of the probe, so as to deflect the nose assembly within the patient's body by an angle in the range 0° to 60° with respect to an inline position along the main length direction of the probe.

A mechanism for adjusting the angle of the nose comprises an actuation wire, preferably in the form of a nylon filament, which is attached to the nose piece and which extends along the tube 8, and can be pulled by means of a hand operated adjustment wheel 13. Operation of the wheel in a first rotational direction causes tension on the cable and pulls the cable so as to deflect the end of the probe from its normal straight ahead bias position, when under no tension from the cable. Rotation of the wheel in an opposite direction causes the cable to pay out and move back along the probe, so the end of the probe can move back towards its normal unbiased straight ahead position. The position of the wheel can be locked using a friction pad, latch or ratchet, to allow the nose to be kept ant any angular deflection of the nose relative to the main probe length, within its maximum range of deflection, so once set, the operator can leave the probe at a selected angle of deflection. Typically there may be markings on the wheel to indicate angles of deflection of the probe end of 0°, 30°, 40°, 50° and 60°.

In other embodiments, the angular adjustment wheel may be replaced by a cam or pulley attached to a filament, wire or cable.

In other embodiments there may be is provided a rack located internally of the body member which is adapted to be displaced manually to and fro along the body member to allow for angular displacement of the end of the nose relative to the inline or co-axial direction of the probe.

The leaf spring 606 biases the bayonet socket to remain in an inline or co-axial position, unless the angle is deflected via the manually operated nose adjuster wheel.

As shown in FIG. 8 herein, when the end of the probe is in a straight ahead position, the strip or leaf spring 606 when in a neutral position as set by the adjustment wheel 10, adopts a straight ahead inline position with the rest of the tube 600. In this configuration, neither the leaf spring 606 nor the exposed side 603 of the tube wall are under tension, and the tube adopts its normal as manufactured straight ahead position.

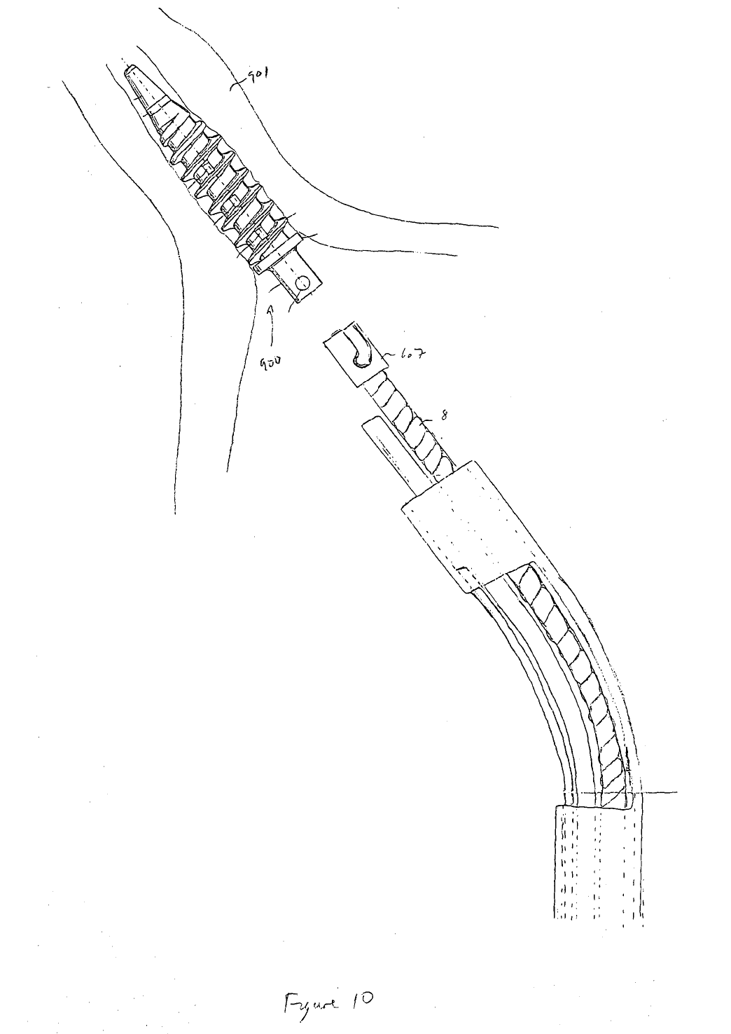

Referring to FIG. 9 herein, when the leaf spring 606 is pulled under tension by the adjustment wheel 10 so as to be drawn back into the tube slightly, the leaf spring pulls the loop end 605 of the tube thereby deflecting the end of the tube from its normal rested straight ahead position. One side of the loop 605 is pulled by the leaf spring 606, so as to bend over the end, about the flexible tube side wall 603, which bends correspondingly. The flexible drive shaft 8 also bends, as does the multi-function cable 600. In this position, the drive shaft 8 is still rotatable either clockwise or anti-clockwise. The flexible drive shaft 8 can rotate the plug clockwise or anticlockwise whilst the nose is in a deflected position.

Also shown in FIG. 9 for illustrative purposes, in cut away view is an ostium 900 and fallopian tube 901 of the female body, showing fitment of the occlusive plug in situ in the patient's body, as the occlusive plug has been driven into the fallopian tube via the ostium, moving itself forward using the self-tapping screw of the occlusive plug. The thread of the plug lodges into the endosalpinx and cuts into the endosalpinx which slightly traumatizes the tissue.

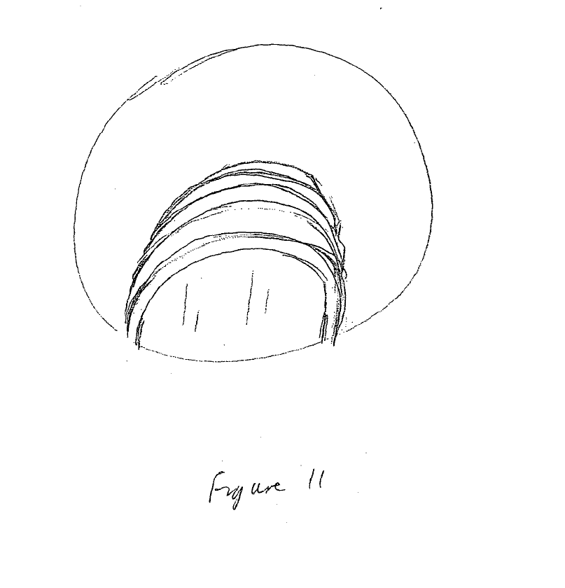

Referring to FIG. 10 herein, there is shown the occlusive plug in situ in the fallopian tube, and detached from the nose 6 of the applicator. Using the stepper motor control, the drive shaft 8 and end connector 608 can be rotated or vibrated in an anti-clockwise or clockwise direction to aid detachment of the end of the occlusive plug from the end connector or the applicator once the occlusive plug is in position.

Operation of the Applicator

The applicator is deployed as follows. The applicator is prepared for use by inserting new batteries into the light source 5, checking operation of the probe adjuster 10 by turning the thumb wheel, connecting power to the power socket 11, and connecting a fluid supply to the fluid connector 12. The practitioner also checks sliding of the sheath backwards and forwards along the probe, and fits an occlusive plug into the connector at the end of the probe. The sheath is slid over the nose of probe, protecting the occlusive plug.

The practitioner inserts the probe into the female patient's vagina, and at the same time may view the end of the probe through the eye piece 3 and optics, under illumination from the light source. The end of the probe is inserted through the neck of the womb, which needs to be dilated. The sheath of sleeve 700 protects plug on the end of the probe. The practitioner may inject fluid via the fluid connector, which flows out of the end of the fluid pipe in the region around the nose. The fluid distends the uterine cavity so that the operator can see inside the cavity and identify for example the left ostium.

Preferably, the eye piece is adapted so that a television camera can be fitted to the eye piece, allowing the practitioner to see the view through the end of the probe on a known close circuit television system, which is ergonomically more efficient for the surgeon, allowing the surgeon to stand upright whilst manipulating the applicator.

The operator may draw back the sheath, exposing the end of the nose whilst the end of the probe is inside the patient's womb, and may deflect the end of the probe using the deflection control thumb wheel 10, (or in other embodiments, a motor driven preset deflection). Rotation of the thumb wheel in one direction causes deflection of the nose from its straight ahead position, and rotation of the thumb wheel in an opposite direction causes the end of the probe to move towards its normal un-deflected straight ahead position.

The operative identifies the ostium through the viewing system and can manipulate the whole body of the applicator, and deflect the end of the probe to align the occlusive plug with the ostium. The geometry of the probe is designed such that the end of the probe lines up correctly in a direction pointing at the ostium, after insertion through the neck of the womb. The tip of the plug can then be manipulated into the ostium and the medical practitioner can drive the plug in a clockwise direction so that the tip of the plug, which has a self-tapping thread, drives into the end of the fallopian tube whilst being rotated by the rotatable drive shaft 8. Depending on the type of lug used, a predetermined number of rotations may be applied by a single activation of the motor control switch.

In other variants, the speed of rotation may be continuously variable, and the practitioner may drive the plug either in the forward or reverse direction as appropriate under fine speed control of the motor control switch 9.

Once fully inserted, the operative must then detach the plug from the connector 608. However first the practitioner may slightly over-tighten the plug within the fallopian tube, so that the tissue around the ostium compresses at the collar at the end of the plug, and then slightly relax the rotation of the plug before removing the connector. This helps stop any trivial bleeding which may have occurred during insertion of the plug.

Removal of the connector from the end of the plug should be by straight forward withdrawal or pulling of the end of the applicator away from the plug, the plug being retained in the fallopian tube. The dimensions of the connector relative to the dimensions of the end of the plug are selected so that the plug normally stays in place in the connector 608, when being driven clockwise or anti-clockwise, but can easily be slid out of the connector, when the connector is withdrawn in a direction along a main central axis of the plug. However, if there is any sticking of the plug, the operator can apply either clockwise or anti-clockwise rotation of the drive shaft, or in a further mode of operation, the stepper motor is driven to make a series of small angle rotations on a stop-start cycle, with a stop-start duty cycle in the range 5 Hz to 20 Hz, which has the effect of vibrating or shaking the plug free of the end connector 608. A variety of preset rotational or shake cycles are as described herein above.

In the best mode, a single operation of the switch 9 in the forwards direction may be preprogrammed to make between 6 and 9 and preferably 7.5 clockwise revolutions of the drive shaft, followed by one anticlockwise revolution, followed by a shake function to detach the connector from the rear of the plug, (alternate back/forwards step rotations).

FIG. 11 herein illustrates schematically a view down the eyepiece, which may be displayed on a monitor screen, e.g. a TV monitor or computer screen, of an occlusive plug connected to the end of the connector 608, as seen through optical fibre bundle viewing scope. Individual turns of the screw thread of the plug can be seen, and the position of the end of the plug can be judged by the operator.

The applicator can also be used to reverse a sterilized patient who has a pair of occlusive plugs fitted. Reversal of the sterilization operation is achieved by inserting the applicator into the uterine cavity of the womb, as herein above described, but without any plug attached to the applicator, sliding back the protective sheath once the probe end is in the uterine cavity, deflecting the end of the probe to align with the ostium, identifying the end of the plug protruding from the ostium and attaching the connector 608 of the applicator to the protruding plug end by sliding the connector on to the plug end.

The motor control 9 is operated to rotate the drive shaft 8 in an anticlockwise direction so that the rotational movement of the plug within the fallopian tube urges the plug in a direction out of the tube into the lumen. There will be a certain amount of trauma to the cornu where the plug was fitted at the end of the fallopian tube, however this part of the womb has good regenerative capacity, and will heal up, resulting in a patent lumen capable of conducting sperm and fertilized egg, so a return to fertility can be expected.

The probe end is then withdrawn slightly, and straightened whilst still in the uterine cavity, and the previously retracted protective sheath is slid over the distal end of the probe to cover the occlusive plug on the end of the probe and prevents the plug becoming detached and loose in the uterine cavity. The plug, attached to the end of the drive shaft and the connector 608 can then be removed by withdrawal of the probe through the neck of the womb. The fallopian tube on the other side of the womb is treated similarly.

Preferred/Optional Features

Other specific embodiments and/or variations of the best mode embodiment may comprise any one or more of the following features.

The eye-piece is a generally tubular moulding in a synthetic plastic.

The synthetic plastic is of medical grade polycarbonate.

The eye-piece is provided in a first mounting socket/spigot for the mounting of a light source attachment e.g. of stainless steel, and a second mounting socket/spigot for the mounting of a flushing attachment e.g. of stainless steel.

The eye-piece is provided at its end adjacent the body member with a flanged nose moulding e.g. of rubber, attached to the eye-piece by a screw-on end cap.

The eye-piece is attached to the body member as a push-fit, by inserting a leading end of the eye piece into a rear end portion of the body member.

The nose-piece is movably mounted by attachment to the carrier tube via the sidewall portion 603. A leaf spring arrangement biases the nose-piece to an in-line, co-axial location.

An actuation wire is attached to the nose-piece via a bayonet screw, and extends along the carrier to a spool having an integral spur wheel in engagement with a rack located internally of the body member, which rack is adapted to be displaced manually to and fro along the body member by the practitioner e.g. using a thumb to deflect the spring and hence to rotate the plug to a different angular position from in-line, if such deflection is required to assist in locating the leading end of the plug, as a pilot nose, in the fallopian tube.

The rack is provided with an integral thumb piece located externally of the body member.

From its in-line position, the leaf spring may be deflected by the practitioner, such that the plug is at 30°, 40°, 50° or 60° with respect to the in-line position.

For a plug having an hexagonal trailing end e.g. as disclosed in WO/2007/072004, the nose-piece includes an hexagonal drive socket connected by a flexible drive cable housed in a carrier tube extending along the probe to the manually operable drive system for translating linear or arcuate movement of a trigger, into rotational movement via a step-up gearing.

As an alternative to a bayonet type fixing, a hexagonal trailing end and complementary drive socket may be provided to connect the releasable plug-to-the-nose piece.

Irrespective of the drive connection to the plug at the nosepiece, the applicator also incorporates means, operable by the practitioner, to induce a vibration at the drive connection, to aid disconnection after the plug has been successfully inserted.

In an alternative embodiment, the vibration means may comprise a rotatable, out-of-balance body, which could be drivable by a drive shaft or by electric induction. In the best mode, vibration is achieved by drive of the stepper motor.

In one embodiment, a manually operable drive system comprises a gearbox housing at least two spur gearwheels, with a bevel friction drive to a drive head in which an end of a flexible drive cable is secured.

Preferably, the gearwheels provide a 1:6 ratio, whereby translational movement of the trigger from a start position to a stop position produces six revolutions of the flexible drive cable and hence six revolutions of the plug which, if the plug is as described in PCT/GB2009/002450, would advance the plug by some 20 mm along the lumen of the fallopian tube.

The optional gearbox preferably comprises a casing e.g. of medical grade polycarbonate, with integral pivot pins for receiving the gearwheels e.g. of Nylon (delrin), with a closure cover e.g. of medical grade polycarbonate, secured to the casing by two stainless steel screws.

Preferably, the trigger comprises a ‘C’-shaped loop able to receive a finger of the practitioner, and an integral lever operable to rotate the gearing to rotate the drive cable.

In one embodiment, the probe also houses a flushing tube extending back to the eye-piece.

The mechanical connection between the body member and the nose piece may be manually operable.

The mechanical connection between the body member and the nose piece may be power operable.

The rotatable drive means extending between the body member and the nose piece may be manually operable.

The rotatable drive means extending between the body member and the nose piece may be power operable.

Power operation is preferably by a battery with electronic controls.

Embodiments of the applicator may be designed to be single use disposable, or engineered to be sterilisable for repeated use in an autoclave.

1-46. (canceled)

47. A medical applicator for use in female sterilisation of a fallopian tube, said applicator comprising:
a body member;
an elongate probe carried by, and projecting from one end of the body member;
a viewing means extending along the probe to an end of the probe distal from the body member;
a connector piece movably mounted at said distal end of the probe;
means for varying the position of the connector with respect to a main length of said probe; and
a rotational drive means extending between the body member and the connector to apply rotational movement to said connector.

48. The applicator as claimed in claim 47, wherein said means for varying the position of said connector comprises a means for actively controlling an angle of connector with respect to a main straight ahead direction of said probe.

49. The applicator as claimed in claim 47, wherein said means for varying the position of the connector comprises a torsion cable for deflecting the end of the probe, and a resilient means for biasing the end of the probe to point in a straight ahead position with respect to the length of the probe, when the end of the probe is not under tension from the torsion cable.

50. The applicator as claimed in claim 47, wherein said means for varying the position of the connector comprises a ring or loop which is moveably mounted to a tube of said probe via a leaf spring arrangement.

51. The applicator as claimed in claim 47, wherein said means for varying the position of the connector with respect to a main length of said probe comprises:
a resilient spring for biasing the end of the probe to an in-line position;
a tension cable for pulling on one side of the end of the probe to deflect the angle of the end of the probe from the its normal un-tensioned straight ahead position; and
a rotatable spool wheel connected to a proximal end of said tension cable,
wherein movement of said spool wheel in a first rotational direction pulls said cable and operates to deflect said probe end from said straight ahead position; and
movement of said spool wheel in a second opposite rotational direction operates to pays out said cable and allows said proximal end of said probe to revert towards said straight ahead position.

52. The applicator as claimed in claim 47, wherein said means for varying the position of the connector with respect to a main length of said probe comprises:
a resilient spring for biasing the end of the probe to an in-line position;
a tension cable for pulling on one side of the end of the probe to deflect the angle of the end of the probe from the its normal un-tensioned straight ahead position; and
a rotatable spool wheel connected to a proximal end of said tension cable,
wherein movement of said spool wheel in a first rotational direction pulls said cable and operates to deflect said probe end from said straight ahead position; and
movement of said spool wheel in a second opposite rotational direction operates to pays out said cable and allows said proximal end of said probe to revert towards said straight ahead position;
and further comprising a latch for releasably locking said spool wheel so as to provide a range of selectable deflection positions of said probe end relative to said straight ahead position.

53. The applicator as claimed in claim 47, further comprising:
a nose piece;
a spool;
an actuation wire which is attached to the nose piece, and extends along the probe to said spool having an integral spur wheel in engagement with a rack located internally of the body member, which rack is adapted to be displaced manually to and fro along the body member to deflect the spring and hence to vary the position of the connector over a range of different angular positions from an inline position of said probe.

54. The applicator as claimed in claim 47, further comprising:
a nose piece;
a spool;
an actuation wire which is attached to the nose piece, and extends along the probe to said spool having an integral spur wheel in engagement with a rack located internally of the body member, which rack is adapted to be displaced manually to and fro along the body member to deflect the spring and hence to vary the position of the connector over a range of different angular positions from an inline position of said probe; and
wherein said rack is provided with an integral thumb piece located externally of the body member.

55. The applicator as claimed in claim 47, comprising a leaf spring arrangement for moving said distal end of said probe relative to a main length direction of said probe, wherein from an inline position of the distal end, said leaf spring may be deflected such that the distal end of the probe is at a 300, 400, 500 or 600 angle with respect to the inline position.

56. The applicator as claimed in claim 47, wherein said viewing means comprises at least one fibre optics cable extending between a proximal end of said probe and a distal end of said probe.

57. The applicator as claimed in claim 47, wherein said viewing means comprises a focussing means for focussing on an end of said fibre optic cable.

58. The applicator as claimed in any claim 47, wherein said viewing means is attached to said body member as a push fit, by inserting a leading end of the viewing means into a rear end portion of said body member.

59. The applicator as claimed in claim 47, wherein said rotational drive means comprises a helical wound flexible drive shaft.

60. The applicator as claimed in claim 47, wherein said connector comprises a bayonet socket connector.

61. The applicator as claimed in claim 47, wherein said rotatable drive means extending between the body member and the nose piece is power operable.

62. The applicator as claimed in claim 47, wherein said rotational drive means comprises a stepper motor.

63. The applicator as claimed in claim 47, wherein said rotational drive means comprises a stepper motor; and a trigger switch for operating said stepper motor, whereby a movement of the trigger from a start position to a stop position produces between 6 and 8 revolutions of a flexible drive cable driven by said stepper motor, and hence 6 to 8 revolutions of the connector for advancing an occlusive plug along the lumen of a fallopian tube.

64. The applicator as claimed in claim 47, wherein said rotatable drive means extending between the body member and the nose piece is manually operable.

65. The applicator as claimed in claim 47, comprising a manually operable drive system comprising a gear box, said gear box housing at least two spur gear wheels with a bevel friction drive to drive a head in which an end of a flexible drive cable is secured.

66. The applicator as claimed in claim 47, wherein said means for varying the position a mechanical connection between said body member and said nose piece is manually operable.

67. The applicator as claimed in claim 47, wherein means for varying the position of the connector with respect to a main length of said probe is power operable.

68. The applicator as claimed in claim 47, further comprising an illumination means for illuminating said distal end of said probe.

69. The applicator as claimed in claim 68, further comprising an illumination means for illuminating said distal end of said probe, wherein said illumination means comprises at least one light conducting optical fibre extending along said probe, for illuminating said distal end of said probe.

70. A medical applicator for use in the fitting of an occlusive plug of the screw in type to a fallopian tube, said applicator comprising:
an elongate probe member, said probe member comprising:
an elongate hollow tubular member;
an elongate flexible drive shaft extending along said tubular member to a distal end of said probe;
wherein said hollow tubular member, comprises at a distal end, a deflectable nose portion which can be controllably directed in a direction making an angle in the range 0 to 60 degrees to a main length direction of said probe.

71. The applicator as claimed in claim 70, wherein said deflectable nose portion comprises a tubular ring member, said tubular ring member being connected to said hollow tubular member by a resilient bendable member; and
a tension cable attached to said tubular ring member, whereby pulling of said tension member causes said ring member to deflect away from main central axis direction of said hollow tube member, so as to bend said distal end of said probe.

72. The applicator as claimed in claim 70, wherein said deflectable nose portion comprises a tubular ring member, said tubular ring member being connected to said hollow tubular member by a resilient bendable member; and
a tension cable attached to said tubular ring member, whereby pulling of said tension member causes said ring member to deflect away from main central axis direction of said hollow tube member, so as to bend said distal end of said probe;
wherein said resilient member comprises a strip leaf spring.

73. The applicator as claimed in claim 70, wherein said flexible drive shaft comprises a helical wound tube.

74. The applicator as claimed in claim 70, wherein said flexible drive shaft comprises a helical spring.

75. The applicator as claimed in claim 70, further comprising a tubular pipe extending within said hollow tubular member, for delivery of fluid to a position at the distal end of said probe.

76. The applicator as claimed in claim 70, comprising at least one fibre optic cable extending along said hollow tube, for illuminating a region at a distal end of said tube.

77. The applicator as claimed in claim 70, further comprising at least one fibre optic cable for conducting a view at a distal end of said hollow tube, to a location at a near end of said hollow tube, said fibre optic cable located inside said hollow tube.

78. An applicator for use in the fitting of an occlusive plug of the screw in type to a fallopian tube, said applicator comprising:
an elongate probe member;
a drive shaft extending along said probe member;
an electric stepper motor connected to said drive shaft; and
a controller for controlling operation of said stepper motor;
wherein said electric stepper motor is configured to drive said drive shaft in a clockwise or an anti-clockwise direction.

79. The applicator as claimed in claim 78, wherein said controller is capable of driving said stepper motor in a series of stop-start rotational steps in a plurality of pre-determined angular movements, at a pre-determined frequency of movement.

80. The applicator as claimed in claim 78, wherein said controller is capable of driving said stepper motor with a continuously variable range of rotational speeds over a pre-determined range of speeds.

81. An applicator for use in the fitting of an occlusive plug said applicator comprising:
an elongate probe;
a connector at a distal end of said probe, for connecting to an occlusive plug;
a fluid tube extending along said probe for carrying fluid to a distal end of said probe;
a fibre optic viewing cable extending along said probe for viewing at a distal end of said probe from a viewing position at a near end of said probe;
a fibre optic light transmission cable for conducting light from said near end of the probe to said distal end of the probe for illuminating said distal end of said probe;
wherein said fluid channel, said fibre optic viewing cable and said fibre optic light transmitting cable are all formed into a single unitary flexible cable extending along a length of said probe.

82. A method of using an applicator, said applicator comprising:
a body member;
an elongate probe carried by, and projecting from one end of the body member;
a viewing means extending along the probe to an end of the probe distal from the body member;
a connector piece movably mounted at said distal end of the probe;
means for varying the position of the connector with respect to a main length of said probe; and
a rotational drive means extending between the body member and the connector to apply rotational movement to said connector,
said method comprising:
fitting an occlusive plug to said connector;
inserting said probe into a neck of a womb;
deflecting said distal end of said probe to point towards a cornu region;
using said viewing means to view a region around said distal end of said probe;
locating a forward end of said occlusive plug at an ostium;
operating said rotational drive means to rotate said connector a predetermined number of revolutions to drive said occlusive plug into said ostium; and
detaching said connector from said occlusive plug.

83. The method as claimed in claim 82, wherein said step of detaching said connector from said occlusive plug comprises driving said rotational drive means in a repeated start-stop rotational movement in a rotational direction opposite to that used to drive the plug into said ostium.

84. The method as claimed in claim 82, comprising viewing a distal end of said probe using a television camera and monitor.

85. The method as claimed in any one of claim 82, comprising introducing a fluid along said probe to a region around said distal end of said probe.

86. A method of female sterilisation using an elongate uterine probe, said uterine probe comprising:
(i) a viewing means extending along the probe to a distal end of the probe;
(ii) a connector piece movably mounted at said distal end of the probe for connecting to an occlusive plug;
(iii) means for varying the position of the connector with respect to a main length of said probe; and
(iv) a rotational drive means operable to apply rotational movement to said connector;
said method comprising:
fitting an occlusive plug to said connector at said distal end of said probe;
inserting said probe into a neck of a womb;
deflecting said distal end of said probe within a uterine cavity, to point towards a cornu region;
using said viewing means to view a region around said distal end of said probe;
locating a forward end of said occlusive plug at an ostium;
operating said rotational drive means to rotate said connector to drive said occlusive plug into said ostium; and
detaching said connector from said occlusive plug.

87. The method as claimed in claim 86, comprising sliding a sheath cover over said occlusive plug at said distal end of said probe after fitting said occlusive plug and prior to inserting said probe through said neck of a womb.

88. The method as claimed in claim 86, wherein said step of detaching said connector from said occlusive plug comprises shaking said connector by applying a series of sharp rotational movements using said rotational drive means.

89. A method of reversing a female sterilisation caused by an occlusive plug positioned in a fallopian tube of a female, using a uterine probe, said probe comprising:
(i) a viewing means extending along the probe to a distal end of the probe;
(ii) a connector piece movably mounted at said distal end of the probe for connecting to an occlusive plug;
(iii) means for varying the position of the said distal end of the probe with respect to a main length of said probe; and
(iv) a rotational drive means operable to apply rotational movement to said connector;
said method comprising:
inserting said probe into a neck of a womb;
deflecting said distal end of said probe within a uterine cavity, to point said distal end towards a cornu region;
using said viewing means to view a region around said distal end of said probe to locate an end of an occlusive plug protruding from an ostium;
locating said connector at said protruding end of said occlusive plug and attaching said connector to said protruding end;
operating said rotational drive means to rotate said connector to withdraw said occlusive plug from said ostium.

90. The method as claimed in claim 89, further comprising withdrawing said probe form said uterine cavity, with said occlusive plug attached to said connector.

91. The method as claimed in claim 89, further comprising: after withdrawing said occlusive plug from said ostium, straightening said distal end of said probe from said deflected attitude prior to withdrawing said probe from said uterine cavity, with said occlusive plug attached.

92. The method as claimed in claim 91, further comprising: after withdrawing said occlusive plug from said ostium, straightening said distal end of said probe from said deflected attitude prior to withdrawing said probe from said uterine cavity, with said occlusive plug attached, and further comprising sliding a cover sheath over said distal end of said probe prior to said withdrawal of the probe from the uterine cavity.
